(19) United States
(12) Patent Application Publication
(10) Pub. No.: US 2019/0254313 A1
(43) Pub. Date: Aug. 22, 2019

(54) FRUIT AND VEGETABLE JUICE RICH IN SOD AND PROCESSING METHOD THEREOF

(71) Applicant: China Agricultural University, Haidian District, Beijing (CN)

(72) Inventors: Xiaojun Liao, Beijing (CN); Zhiqiang Hou, Beijing (CN); Liang Zhao, Beijing (CN); Yongtao Wang, Beijing (CN)

(73) Assignees: China Agricultural University, Haidian District, Beijing (CN); China Agricultural University, Haidian District, Beijing (CN)

(21) Appl. No.: 16/090,559

(22) PCT Filed: Mar. 31, 2016
(86) PCT No.: PCT/CN2016/077937

§ 371 Date: Oct. 1, 2018

(30) Foreign Application Priority Data
Mar. 21, 2016 (CN) 201610162004.9

(51) Int. Cl. A23L 2/02; A23L 3/015; C12N 9/02

(52) U.S. Cl. CPC A23L 2/02; A23L 3/0155; A23V 2300/46; A23V 2002/00; A23V 2200/30; C12N 9/0089

(57) ABSTRACT

A method of forming a fruit and vegetable juice rich in superoxide dismutase (SOD) comprises subjecting the fruit and vegetable juice to ultra-high-pressure processing, the pressure is 100 MPa to 800 MPa, and the time period is 1 minute to 30 minutes. After processings with the present method, the enzymatic activity of SOD is stable and, at the same time, the microorganisms in the fruit and vegetable juice can be killed. Thus, fruit and vegetable juices rich in superoxide SOD may be provided thereby.

### CROSS-REFERENCE TO RELATED APPLICATIONS

This application is a national phase entry under 35 U.S.C. § 371 of International Patent Application PCT/CN2016/077937, filed Mar. 31, 2016, designating the United States of America and published as International Patent Publication WO 2017/161600 A1 on Sep. 28, 2017, which claims the benefit under Article 8 of the Patent Cooperation Treaty to Chinese Patent Application Serial No. 201610162004.9, filed Mar. 21, 2016.

### TECHNICAL FIELD

This application relates to the field of biotechnology, in particular, to a fruit and vegetable juice rich in superoxide dismutase (SOD) and a processing method thereof.

### BACKGROUND

Superoxide dismutase (SOD) is a type of oxidoreductase widely found in various organisms. Superoxide dismutase is a metalloenzyme capable of removing superoxide radical anion in the body and effectively preventing the toxic effect of superoxide radical anion on the body, and is a widely used medicinal enzyme.

So far, SOD has been isolated from organisms such as bacteria, fungi, protozoa, algae, insects, fish, plants and mammals. Due to the differences in metal prosthetic groups, these SODs can be classified into at least four types: Cu/Zn-SOD, Mn-SOD, Fe-SOD, and Ni-SOD. Plant-derived SOD has been shown to have a positive effect on human health. Common fruits and vegetables rich in SOD enzymes include sea-buckthorn, Rosa roxburghii, kiwifruit, mulberry, etc., and are often used to extract SOD enzymes or processed into health products.

Sea-buckthorn is mainly distributed in the northeast region, the north region, the northwest region and the southwest region in China. In modern studies, it has been reported that sea-buckthorn fruit contains many nutritive active substances and nutrients, has an extremely high Vc content of about 1000 mg to 1600 mg/100 g, which is 105 times the content found in apple, and is called “the King of Vc.” In addition, sea-buckthorn fruit is abundant in V_{E}, carotene, flavonoids and phenolic substances, such that sea-buckthorn fruit has strong anti-oxidation and healthcare functions. The activity and content of SOD enzyme in sea-buckthorn fruit are high, and SOD is a very important efficacy factor for anti-oxidation effect of sea-buckthorn.

Sea-buckthorn fruit contains such abundant bioactive substances and nutrients, indicating that the sea-buckthorn has great nutritional value for the human body. Sea-buckthorn fruit has anti-tumor effect and good protective effect on the cardiovascular system. At the same time, sea-buckthorn can also promote the increase of animal saliva, gastrointestinal gland secretion and the content of pepsin, and has a stimulating effect on gastrointestinal motility, which is beneficial to food digestion and absorption. The active ingredients of sea-buckthorn have the effect in eliminating free radicals in the human body and blocking peroxidation, and delaying human aging. Therefore, sea-buckthorn fruit is widely used in the fields such as medicine, food, beverages, cosmetics and the like, and has a very high economic value.

Rosa roxburghii is a wild plant of the Rosaceae, and is mostly distributed in Guizhou province, Sichuan province, Yunnan province, Shaanxi province, etc. The sarcocarp of Rosa roxburghii is crisp, sweet and sour; in addition to fresh eating, it is a high-quality raw material for processing advanced beverages. Rosa roxburghii contains abundant Vc; organic acids such as malic acid, lactic acid, tartaric acid, citric acid; polyphenols and polysaccharides. In addition, Rosa roxburghii fruit contains abundant β-sitosterol and SOD enzymes, and has good healthcare functions.

Rosa roxburghii fruit and fruit juice have been proven to have good effect in resisting oxidation, eliminating free radicals in the body, and delaying organism aging, and have certain effects in preventing cardiovascular diseases and cancer. The components of organic acids contained in Rosa roxburghii are not only flavor substances, but also important Chinese herbal medicinal ingredients, and can promote digestion and acid-base balance in the human body. The sterols extracted from Rosa roxburghii have strong anti-inflammatory effects, and the effect of inhibiting the absorption of cholesterol by the human body, promoting the degradation and metabolism of cholesterol, and inhibiting the biochemical synthesis of cholesterol and the like. Rosa roxburghii can be used to prevent and treat heart disease of coronary atherosclerosis type.

Sea-buckthorn and Rosa roxburghii, which are rich in SOD enzymes and nutrients, are often exploited to produce fruit and vegetable juice products. Although conventional heat processing methods can effectively kill microorganisms and ensure the product safety, these methods may lead to inactivation of SOD enzymes and loss of healthcare function during processing, and thus such methods are not suitable for producing products with high SOD enzyme activity.

### BRIEF SUMMARY

In order to solve the above technical problem, the present disclosure provides a fruit and vegetable juice rich in SOD and a processing method thereof. The SOD enzyme activity is stabilized after being treated by the method of the present disclosure, the healthcare function of the product is improved, and the problem of the inactivation of SOD enzyme during processing is effectively solved. At the same time, microorganisms in oral liquids can be killed, which ensures product safety and prolongs shelf life. The products obtained by the method of the present disclosure are more beneficial to human health.

The technical solutions of the disclosure are provided as follows:

A processing method of fruit and vegetable juice rich in SOD comprises subjecting the fruit and vegetable juice to ultra-high-pressure processing under the following conditions: a pressure of 100 MPa to 800 MPa; and a time period of 1 minute to 30 minutes.

Preferably, the conditions of the ultra-high-pressure processing are as follows: a pressure of 100 MPa to 600 MPa; and a time period of 1 minute to 15 minutes.

It is found in the study that under the conditions of the above-mentioned ultra-high-pressure processing, microorganisms can be effectively killed, product safety can be ensured, shelf life can be prolonged, the stability of the SOD activity in the fruit and vegetable juice can be maintained, and the healthcare function of the product can be ensured.

It is surprisingly found that, when the ultra-high-pressure processing is carried out under the pressure of 300 MPa to 500 MPa for a time period of 4 minutes to 10 minutes, especially when carried out under the pressure of 500 MPa and a time period of 5 minutes, the activity of SOD in fruit and vegetable juice can be significantly increased, thereby greatly improving the healthcare function of the product.

Further, it is found in the study that, when the pressure of the above-mentioned ultra-high-pressure processing is controlled at 500 MPa and the time period is controlled to 10 minutes or more, it is more beneficial to prolong the shelf life of the product.

In general, the above-mentioned ultra-high-pressure processing is carried out at room temperature, and the temperature is in the range of 15° C. to 50° C.

The pressure-transmitting medium of the ultra-high-pressure processing of the present disclosure is preferably water.

The raw material of the fruit and vegetable juice of the present disclosure may use common fruits and vegetables, preferably one or more of fruits and vegetables rich in SOD, such as Rosa roxburghii, sea-buckthorn, kiwifruit, and mulberry.

The fruit and vegetable juice before the ultra-high-pressure processing can be prepared by conventional methods in the art.

It is found in the study that, in the process of preparing the fruit and vegetable juice, when the percentage of the raw materials with a mechanical damage area greater than 2% is controlled to be less than 1% of the total raw materials, it is more beneficial to obtain the fruit and vegetable juice rich in SOD. After the above-mentioned ultra-high-pressure processing, it is more beneficial to maintain the stability of SOD activity in the fruit and vegetable juice, and to prolong the shelf life. When the ultra-high-pressure processing is carried out under the conditions of 500 MPa for a time period of 5 minutes, the fruit and vegetable juice can be stored at 4° C. for 2 months, and the shelf life is significantly longer than that of the fruit and vegetable juice prepared by the traditional thermization (105° C., and 15 seconds).

In the process of preparing the fruit and vegetable juice, the fruits and vegetables are subjected to pulping; preferably, the amount of water added in the pulping process is 1 to 3 times, more preferably 2 times, the weight of the fruits and vegetables.

The present disclosure also provides the fruit and vegetable juice rich in SOD prepared according to the above-mentioned method.

Preferably, the fruit and vegetable juice rich in SOD has a soluble solid content of 3° to 6° Brix and a pH of 2.8-3.5.

High-Pressure Processing means that the food placed in the ultra-high-pressure processing chamber is treated with a pressure of 100 MPa to 1000 MPa under room temperature or under mild heating conditions, using water or other liquid as pressurizing medium; after the pressure rise is finished, the food is statically placed for a certain period of time at the point of the setting maximum pressure, so as to kill microorganisms and ensure food safety.

The present disclosure has the following beneficial effects:

1) The present disclosure adopts an ultra-high-pressure processing method instead of the traditional thermization processing. The present method not only effectively kills microorganisms, ensures product safety, and prolongs the shelf life, but also maintains the stability of SOD activity in the fruit and vegetable juice, and ensures the healthcare function of the product. The inactivating effect of traditional heat processing methods on SOD enzyme is effectively avoided.

2) Especially under specific conditions of the ultra-high pressure, SOD enzyme activity in fruit and vegetable juice can be significantly improved, thereby greatly enhancing the healthcare function of the product.

3) The ultra-high-pressure processing of the present disclosure adopts water as a pressure-transmitting medium, which will not cause contamination to the product; it is a green, clean and environmentally friendly processing method; and the energy consumption is low, which is beneficial to cost savings.

### BRIEF DESCRIPTION OF THE DRAWINGS

FIG. 1 shows the changes in the total plate count in the fruit and vegetable juice of Example 4 and Comparative Example 2 during storage.

FIG. 2 shows the changes in the total plate count in the fruit and vegetable juice of Example 8 and Comparative Example 4 during storage.

### DETAILED DESCRIPTION

The following examples are intended to illustrate the present disclosure but not to limit the scope thereof. Where the specific techniques or conditions are not indicated in the Examples, they are performed according to the techniques or conditions described in the literature in the field or according to the product specifications. Where the manufacturers of the reagents or instruments used are not indicated, they are regular products that can be purchased through regulated channels.

### EXAMPLE 1

A processing method of sea-buckthorn juice rich in SOD comprising the following steps: the sea-buckthorn juice was subjected to ultra-high-pressure processing under the conditions of a pressure of 400 MPa for a time period of 5 minutes. The specific method comprises the following steps: the sea-buckthorn juice was dispensed into 30 ml PET bottles, then the lid was covered tightly, and the ultra-high-pressure processing was performed under the above conditions, with water as the pressure-transmitting medium.

Wherein, the processing method of the sea-buckthorn juice comprises the following steps:

(1) fresh sea-buckthorn was selected;

(2) the selected sea-buckthorn was cleaned;

(3) the sea-buckthorn was fed into a pulper, and water equivalent in weight to the sea-buckthorn was added to perform pulping; and

(4) the resulting mixture was filtered by four layers of gauze so as to separate the pulp from the pips and residues of pericarps to obtain sea-buckthorn juice.

This Example also provided a sea-buckthorn juice rich in SOD prepared according to the method, which has a soluble solid content of about 3.6° Brix and a pH of 2.8.

### EXAMPLE 2

A processing method of sea-buckthorn juice rich in SOD, which was carried out in the same manner as that in Example 1 except that the ultra-high-pressure processing was carried out under the conditions of a pressure of 500 MPa and a time period of 5 minutes.

### EXAMPLE 3

A processing method of sea-buckthorn juice rich in SOD, which was carried out in the same manner as that in Example 1 except that the ultra-high-pressure processing was carried out under the conditions of a pressure of 300 MPa and a time period of 5 minutes.

### EXAMPLE 4

A processing method of sea-buckthorn juice rich in SOD, which was carried out in the same manner as that in Example 1 except that the ultra-high-pressure processing was carried out under the conditions of a pressure of 500 MPa and a time period of 10 minutes.

### EXAMPLE 5

A processing method of Rosa roxburghii juice rich in SOD comprises the following steps: the Rosa roxburghii juice was subjected to ultra-high-pressure processing under the conditions of a pressure of 400 MPa for a time period of 5 minutes. The specific method is comprised of the following steps: the Rosa roxburghii juice was dispensed into 30 ml PET bottles, then the lid was covered tightly, and the ultra-high-pressure processing was performed under the above conditions.

Wherein, the processing method of the Rosa roxburghii juice comprises the following steps:

(1) fresh Rosa roxburghii was selected, and the content of the Rosa roxburghii with a mechanical damage area greater than 2% in the total raw materials was controlled within 1%;

(2) the selected Rosa roxburghii was cleaned;

(3) the Rosa roxburghii fruit was fed into a pulper, and water equivalent in weight to Rosa roxburghii was added to perform pulping; and

(4) the resulting mixture was filtered by four layers of gauze so as to separate the pulp from the pips and residues of pericarps to obtain Rosa roxburghii juice.

This Example also provided a Rosa roxburghii juice rich in SOD prepared according to the method, which has a soluble solid content of about 3° Brix, and a pH of 3.4.

### EXAMPLE 6

A processing method of Rosa roxburghii juice rich in SOD, which was carried out in the same manner as that in Example 5 except that the ultra-high-pressure processing was carried out under the conditions of a pressure of 500 MPa and a time period of 5 minutes.

### EXAMPLE 7

A processing method of Rosa roxburghii juice rich in SOD, which was carried out in the same manner as that in Example 5 except that the ultra-high-pressure processing was carried out under the conditions of a pressure of 400 MPa and a time period of 5 minutes.

### EXAMPLE 8

A processing method of Rosa roxburghii juice rich in SOD, which was carried out in the same manner as that in Example 5 except that the ultra-high-pressure processing was carried out under the conditions of a pressure of 500 MPa and a time period of 10 minutes.

### COMPARATIVE EXAMPLE 1

A processing method of sea-buckthorn juice, which was carried out in the same manner as that in Example 1 except that the ultra-high-pressure processing was carried out under the conditions of a pressure of 600 MPa and a time period of 5 minutes.

### COMPARATIVE EXAMPLE 2

A processing method of sea-buckthorn juice, which was carried out in the same manner as that in Example 1 except for using high-temperature short-time sterilizing treatment (i.e., instead of ultra-high-pressure processing) under the conditions of a temperature of 105° C. and a time period of 15 seconds.

### COMPARATIVE EXAMPLE 3

A processing method of Rosa roxburghii juice, which was carried out in the same manner as that in Example 5 except that the ultra-high-pressure processing was carried out under the conditions of a pressure of 600 MPa and a time period of 5 minutes.

### COMPARATIVE EXAMPLE 4

The present Comparative Example 4 was carried out in the same manner as that in Example 3 except for using a high-temperature short-time sterilizing treatment (i.e., instead of ultra-high-pressure processing) under the conditions of a temperature of 105° C. and a time period of 15 seconds.

### EXPERIMENTAL EXAMPLE 1

The SOD enzyme activities and the microbial indexes of the sea-buckthorn juice prepared in Examples 1 to 4 and Comparative Examples 1 and 2 and the sea-buckthorn juice prepared in Example 1 without undergoing ultra-high-pressure processing were tested, respectively. The results were shown in Table 1 below. The test method for SOD activity was WST-1 method (Reference document: A. V. Peskin and C. C. Winterbourn (2000), “A microtiter plate assay for superoxide dismutase using a water-soluble tetrazolium salt (WST-1),” Clinica Chimica Acta, 293(1-2), 157-166.). WST-1 is 2-(4-iodophenyl)-3-(4-nitrophenyl)-5-(2,4-disulfophenyl)-2H-tetrazolium, monosodium salt. WST-1 can react with superoxide anion generated under the catalysis of xanthine oxidase to produce a water-soluble formazan dye, and the reaction step can be inhibited by SOD. The enzyme activity of SOD could be calculated based on colorimetric analysis of the WST-1 product. With respect to microbial indexes, the microbial colonies were counted according to relative operations of GB 4789.2-2010 “Microbiological examination of food hygiene-Detection of aerobic bacterial count.” The medium for the detection of aerobic bacterial count was plate count agar medium, and culturing was performed at 36° C.±1° C. for 48 hours±2 hours. The molds and yeasts were counted according to the relative operations of GB4789.15-2010 “Microbiological examination of food hygiene—Enumeration of Yeasts and Molds,” rose bengal medium was used, and culturing was performed at 28° C.±1° C. for 5 days.

TABLE 1

| | | Total | |
|---|---|---|---|
| | SOD enzyme | plate count | Molds and |
| | activity U/ml | cfu/ml | yeasts cfu/ml |
| Sea-buckthorn juice of | 702.5 | 8.3 × 10² | 2.2 × 10² |
| Example 1 (without | | | |
| ultra-high-pressure | | | |
| processing) | | | |
| Example 1 | 786.4 | 10 | not detected |
| Example 2 | 896.23 | 11 | not detected |
| Example 3 | 701.40 | 9 | not detected |
| Example 4 | 897.16 | 3 | not detected |
| Comparative Example 1 | 605.28 | 3 | not detected |
| Comparative Example 2 | 223.1 | 6 | not detected |

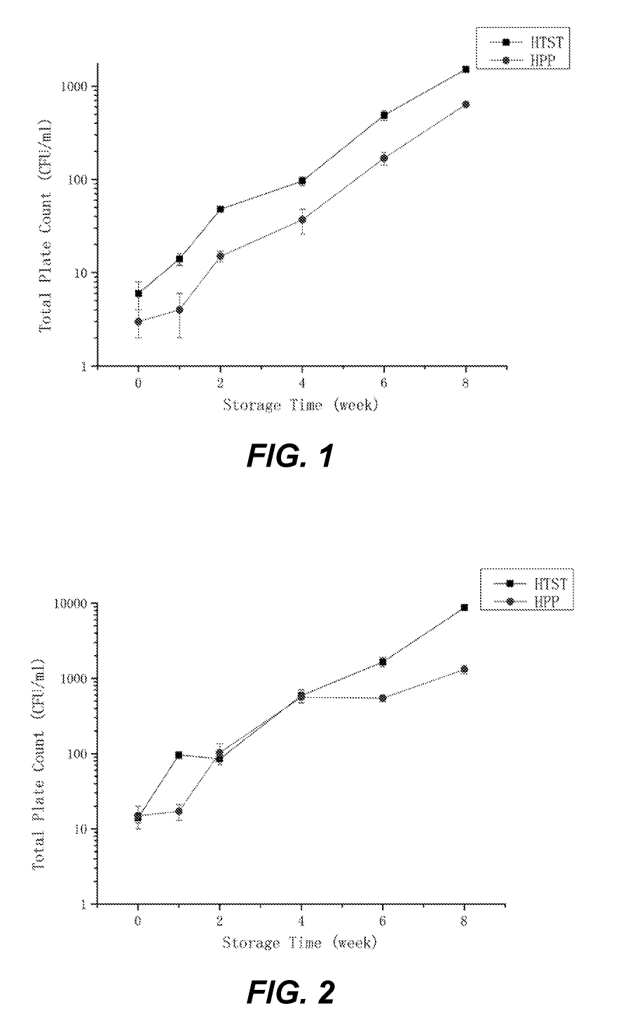

The results in Table 1 showed that all pressure conditions and heat treatment conditions could effectively kill mold and yeast in sea-buckthorn juice. Example 4 and Comparative Example 1 had the best killing effect on indigenous flora. The SOD enzyme activity of Example 1 was slightly improved, the SOD enzyme activity of Example 2 was significantly improved, the SOD enzyme activity of Example 3 could keep relatively stable, the SOD enzyme activity of Example 4 was significantly improved and a shelf life of two months could be guaranteed at 4° C. (see FIG. 1). In Comparative Example 1, SOD enzyme activity in sea-buckthorn juice was reduced because of the ultra-high-pressure condition of 600 MPa. In Comparative Example 2, the high-temperature short-time sterilization decreased the SOD enzyme activity by 65% or more, and the total plate count at 4° C. was higher than that of Example 4 (FIG. 1), indicating that appropriate high-pressure conditions can not only enhance the SOD enzyme activity of sea-buckthorn juice, but also ensure a shelf life of two months and safety of the products.

In FIG. 1, the terms are as follows: HTST: high-temperature short-term treatment, and HPP: ultra-high-pressure processing.

### EXPERIMENTAL EXAMPLE 2

The SOD enzyme activities and microbial indexes of the Rosa roxburghii juice prepared in Examples 5 to 8 and Comparative Examples 3 and 4 and the Rosa roxburghii juice prepared in Example 5 without undergoing ultra-high-pressure processing were tested, respectively. The results are shown in Table 2 below. The detection method for SOD activity and the microorganism detection method were the same as those in Experimental Example 1.

TABLE 2

| | SOD enzyme | Total plate | Molds and |
|---|---|---|---|
| | activity U/ml | count cfu/ml | yeasts cfu/ml |
| Rosa roxburghii juice of | 498.8 | 4.8 × 10⁴ | 3.2 × 10³ |
| Example 5 (without ultra- | | | |
| high-pressure processing) | | | |
| Example 5 | 552.6 | 4 | not detected |
| Example 6 | 685.23 | 32 | not detected |
| Example 7 | 500.13 | 45 | not detected |
| Example 8 | 693.26 | 15 | not detected |
| Comparative Example 3 | 401.28 | 12 | not detected |
| Comparative Example 4 | 162.18 | 14 | not detected |

The results in Table 2 showed that all pressure conditions and heat treatment conditions could effectively kill mold and yeast in Rosa roxburghii juice. Example 5 and Comparative Example 3 had the best killing effect on indigenous flora. The SOD enzyme activity of Example 5 was slightly improved, the SOD enzyme activity of Example 6 was significantly improved, the SOD enzyme activity of Example 7 could keep relatively stable, the SOD enzyme activity of Example 8 was significantly improved and a shelf life of two months can be guaranteed at 4° C. (see FIG. 2). In Comparative Example 3, SOD enzyme activity in Rosa roxburghii juice was reduced because of the ultra-high-pressure condition of 600 MPa. In Comparative Example 4, the high-temperature short-time sterilization decreased the SOD enzyme activity by 65% or more, and the total plate count at 4° C. was higher than that of Example 8 (FIG. 2), indicating that appropriate high-pressure conditions not only enhances the SOD enzyme activity of Rosa roxburghii juice, but also ensures a shelf life of two months and safety of the products.

In FIG. 2, the terms are as follows: HTST: high-temperature short-term treatment, and HPP: ultra-high-pressure processing.

Although the present disclosure has been described above in detail with general description and specific embodiments, it is obvious to a person skilled in the art that some modifications or improvements can be made on the basis of the present disclosure. Therefore, these modifications or improvements made without departing from the spirit of the present disclosure all fall within the protection scope of the present application.

### INDUSTRIAL APPLICABILITY

The disclosure provides a fruit and vegetable juice rich in SOD and a processing method thereof. The method comprises subjecting the fruit and vegetable juice to ultra-high-pressure processing under a pressure of 100 MPa to 800 MPa for a time period of 1 minute to 30 minutes. The SOD enzyme activity is stabilized after being treated by the method of the present disclosure, the healthcare function of the product is improved, and the problem of the inactivation of SOD enzyme during processing is effectively solved. At the same time, microorganisms in fruit and vegetable juice can be killed, which ensures the product safety and prolongs the shelf life. The products obtained by the method of the present disclosure are more beneficial to human health. The present disclosure has a broad application prospect and good industrial applicability in the field of food and health food processing.

1. A processing method of fruit and vegetable juice rich in SOD, characterized in that, (1) the processing method comprises subjecting the fruit and vegetable juice to pulping, the amount of water added in the pulping process is 1 to 3 times of the weight of the fruits and vegetables, (2) subjecting the fruit and vegetable juice to ultra-high pressure processing, in the process of preparing the fruit and vegetable juice, the percentage of the raw materials that have a mechanical damage area of greater than 2% is controlled at less than 1% of total raw materials, the conditions of the ultra-high pressure processing are as follows: a pressure of 500 Mpa, and a time period of 10 min, the raw material of the fruit and vegetable juice is sea-buckthorn, the temperature for the ultra-high pressure processing is in range of 15° C. to 50° C., wherein the fruit and vegetable juice has a soluble solid content of 3 to 6° Brix, and a pH of 2.8 to 3.5.

2. The processing method according to claim 1, characterized in that, the amount of water added in the pulping process is 2 times of the weight of the fruits and vegetables.

3.-10. (canceled)
